(19) United States
(12) Patent Application Publication
(10) Pub. No.: US 2009/0093434 A1
(43) Pub. Date: Apr. 9, 2009

(54) USE OF EIF-5A1 SIRNA TO PROTECT ISLETS CELLS FROM APOPTOSIS AND TO PRESERVE THEIR FUNCTIONALITY

(75) Inventors: John E. THOMPSON, Waterloo (CA); Charles A. Dinarello, Boulder, CO (US)

Correspondence Address: Baker Donelson Bearman, Caldwell & Berkowitz, PC, 555 Eleventh Street, NW, Sixth Floor, Washington, DC, 20004, US

(73) Assignee: Senesco Technologies, Inc., New Brunswick, NJ (US)

(21) Appl. No.: 12/194,686

(22) Filed: Aug. 20, 2008

Related U.S. Application Data
(60) Provisional application No. 60/956,867, filed on Aug. 20, 2007.
(60) Provisional application No. 61/043,074, filed on Apr. 7, 2008.

(51) Int. Cl. A61K 31/7105

(52) U.S. Cl. 514/44

(57) ABSTRACT

The present invention relates to methods for improving the viability, recovery and functionality of islets that are separated from a donor organ for subsequent transplantation and more particularly relates to the use of eIF-5A1 siRNAs to enhance the viability and functionality of islets.

### BACKGROUND OF THE INVENTION

The islets of Langerhans is a multi-cellular entity containing cells that produce insulin within the pancreas. The average person has about a million islets, and they contain approximately two to three percent of the total number of cells in the pancreas. The pancreas contains the islets of Langerhans, which house beta cells that produce insulin. The beta cells monitor glucose levels in the blood and release finely measured amounts of insulin to counterbalance glucose peaks. Type I and II diabetes develop when more than 90 percent of these beta cells are damaged.

Type 1 diabetes is a disorder of glucose homeostasis that affects ˜10% of the 21 million diabetic individuals in the USA. Type 1 diabetes results from the virtually complete auto-immune destruction of pancreatic islet β cells, leaving individuals dependent upon the administration of insulin to maintain life. The pathogenesis of Type 1 diabetes involves a complex interplay between cells of the immune system and antigens present on the islet β cell. This interplay leads to the activation of Th1 cells and macrophages, which subsequently release, among other factors, cytokines (IL1β, TNFα, and IFNγ) that induce nitric oxide production, which triggers both apoptotic and necrotic islet β cell death. β cell “replacement” strategies involving pancreas or islet transplantations have been variably successful in reversing type 1 diabetes, but require tissues from limiting sources (cadaveric donors) and are still subject to destruction by the underlying autoimmune process.

Separation or isolation of the islets from the connective matrix and remaining exocrine tissue is advantageous and beneficial for laboratory experimentation and transplantation purposes. Islet transplantation is a most promising and minimally physiologically invasive procedure for treatment of Type I diabetes mellitus. Transplanting islets rather than complete pancreatic tissue has the distinct advantages of ease of transplantation, and the elimination of the pancreatic exocrine function of the donor tissue involving secretion of digestive enzymes. Liberating islets from pancreatic exocrine tissue is the initial and crucial step that influences islet transplantations. The important objective in islet isolation is to provide sufficient numbers of viable functional and potent islets for transplantation.

The “Edmonton Protocol” transplants healthy islets into diabetic patients. Islet transplantation using the Edmonton Protocol is described in Shapiro, Ryan, and Lakey, Clinical Islet Transplantation—State of the Art, Transplantation Proceedings, 33, pp. 3502-3503 (2001); Ryan et al., Clinical Outcomes and Insulin Secretion After Islet Transplantation With the Edmonton Protocol, Diabetes, Vol. 50, April 2001, pp. 710-719; and Ryan et al., Continued Insulin Reserve Provides Long-Term Glycemic Control, Diabetes, Vol. 51, July 2002, pp. 2148-2157. Once in the liver, the cells develop a blood supply and begin producing insulin. The Edmonton Protocol may include 7-10 steps depending on the method employed. The first step involves the delivery of a specific enzyme (liberase) to a donor pancreas, which digests the pancreas tissue, but does not digest the islets. Following the digestion step, there are several successive steps for separating the islets from other cells in the pancreas. The separated islets are transplanted into the main vessel of the liver, known as the portal vein. The liver is able to regenerate itself when damaged, building new blood vessels and supporting tissue. Therefore, when islets are transplanted into the liver, it is believed that new blood vessels form to support the islets. The insulin that the cells produce is absorbed into the blood stream through these surrounding vessels and distributed through the body to control glucose levels in the blood.

Altogether, the steps of the Edmonton Protocol create a vigorous process that compromises the viability of islets, which have a fragile, three-dimensional structure and require large amounts of oxygen for growth and viability. During the process, islets may be damaged or destroyed due to non-optimal conditions of oxygen delivery, affecting the yield of healthy islets that are retrieved from a given donor pancreas. Furthermore, islet transplantation is severely limited by donor availability; frequently, two pancreata are required to obtain insulin independence in just one patient.

Islet transplantation, together with steroid-free, nondiabetogenic immunosuppressive therapy, has been used to treat patients with Type 1 diabetes. However, such treatments can lead to increased risk of hyperlipidemia and hypertension, and long-term studies demonstrate that islet viability is impaired.

The Edmonton protocol of islet transplantation in humans has demonstrated remarkable short term success, with 80% of individuals achieving insulin independence at 1 year post-transplant. However, this rate decreases to only about 10-15% at 5 years. The reasons for this progressive loss of graft function is unclear, but likely includes islet loss as a result of ongoing cytokine-mediated inflammation. However, islet transplantation may continue to remain a viable therapeutic option for individuals with Type 1 diabetes, particularly as results show that even those that fail long-term insulin independence, they still retain other important benefits, such as decreased blood sugar lability, lower incidence of hypoglycemia, and decreased insulin dosage.

The first days following islet transplantation are a particularly vulnerable period for the transplanted islets. Up to 60% of transplanted islets undergo non-autoantigen-specific apoptosis in the first days following transplantation. This early destruction is the major underlying reason for requiring the use of two to three cadaveric donors to ensure sufficient islets survive to reverse diabetes. Local production of inflammatory cytokines IL-1β, TNFα, and IFNγ have been implicated in the early apoptosis of transplanted islets. Thus, strategies that limit cytokine production in the early transplantation period may both limit the number of donors necessary to obtain sufficient islets, and also enhance islet survivability and prolong their ability to function in the long term. The present invention provides these strategies.

### SUMMARY OF THE INVENTION

The present invention provides a method for preserving the functionality of harvested islet cells after isolation comprising administering eIF-5A1 siRNA to the islet cells of an islet cell donor prior to islet isolation, wherein the eIF-5A1 siRNA inhibits expression of eIF-5A1 in the islet cells and thereby inhibits apoptosis in the islet cells and preserves the functionality of the harvested islet cells. In preferred embodiments the siRNA targets the following nucleotide sequences of eIF-5A1: 5′-AAAGGAATGACTTCCAGCTGA-3′; 5′-AAGATCGTCGAGATGTCTACT-3′; 5′-AAGGTCCATCTGGTTGGTATT-3′; or 5′-AAGCTGGACTCCTCCTACACA-3′. In certain embodiments the eIF-5A1 siRNA comprises the nucleotide sequence 5′-AAAGGAAUGACUUCCAGCTGAdTdT-3′. The siRNA may be administered to the donor through any acceptable means. In certain embodiments, the siRNA is administered to the islet cell donor via intraperotoneal injection.

Another embodiment of the present invention provides a method for inhibiting islet cells from undergoing apoptosis during a donor harvesting process comprising administering eIF-5A1 siRNA to an islet cell donor via intraperotoneal injection to islet cell donor prior to islet isolation, wherein the eIF-5A1 siRNA inhibits expression of eIF-5A1 in the islet cells and thereby inhibits apoptosis in the islet cells.

In preferred embodiments the siRNA targets the following nucleotide sequences of eIF-5A1: 5′-AAAGGAATGACTTCCAGCTGA-3′; 5′-AAGATCGTCGAGATGTCTACT-3′; 5′-AAGGTCCATCTGGTTGGTATT-3′; or 5′-AAGCTGGACTCCTCCTACACA-3′. In certain embodiments the eIF-5A1 siRNA comprises the nucleotide sequence 5′-AAAGGAAUGACUUCCAGCTGAdTdT-3′.

Any siRNA or antisense construct can be used, as long as such construct inhibits expression of eIF-5A1 in the islet cells. Administration of siRNA may be by any suitable route. Exemplary administration methods include perfusion through the portal vein of the islet cell donor, hydrodynamic perfusion through the portal vein of the islet cell donor, and intraperotoneal administration.

The present invention also provides a composition for inhibiting apoptosis and preserving functionality of in islet cells, comprising eIF-5A1 siRNA, wherein the siRNA inhibits expression of eIF-5A1 and thereby inhibits apoptosis in the islet cells. Preferred eIF-5A1 siRNAs are discussed above.

The present invention also provides a method of inhibiting expression of a target protein in islet β cells comprising administering to a β cell donor prior to β cell harvesting an siRNA construct targeted to the mRNA encoding the target protein, wherein the siRNA inhibits expression of the target protein in the islet β cells.

The invention also provides a method of decreasing the production of nitric oxide (NO) via the inducible nitric oxide synthase (iNOS) pathway, by decreasing expression of eIF-FA.

The invention also provide the use of eIF5A siRNA to manufacture a medicament to decrease production of nitric oxide via the iNOS pathway.

### BRIEF DESCRIPTION OF THE DRAWINGS

FIG. 1 provides results of RT-PCR performed for β-actin, mAAT and eIF-5A1 after perfusion through the portal vein with eIF-5A siRNA. This figure shows that eIF-5A1 expression is measurable and was thus incorporated into islets.

FIG. 2 shows slows retrograde portal vein perfusion. Bile duct (clear) and portal vein (red) ready for preparatory knot (dark suture). The needle enters below the knot (direction indicated by arrow), cross under the knot and releases siRNA into vessels that reach pancreas, spleen, intestine and a third of distal colon.

FIG. 3 shows that perfusion of eIF-5A1 siRNA into islets causes a reduction of expression of eIF-5A1 (shown is reduction in mRNA levels of eIF-5A1).

FIG. 4 shows a reduction of apoptosis of islets cells having been treated with eIF5-5A1 siRNA as compared to control and saline treated islets (here n=2 per group).

FIG. 5 shows a reduction of apoptosis of islets cells having been treated with eIF5-5A1 siRNA as compared to control and saline treated islets (here n=3 per group).

FIG. 6 provides the nucleotide sequence of human eIF-5A1 aligned against eIF5-A2.

FIG. 7 provides the amino acid sequence of human eIF-5A1 aligned against eIF5-A2.

FIG. 8 provides the nucleotide sequence of human eIF-5A1 with exemplary antisense oligonucleotides.

FIG. 9 provides the nucleotide sequence of human eIF-5A1 with exemplary antisense oligonucleotides.

FIGS. 10A and B provide the nucleotide sequence of human eIF-5A1 with exemplary siRNAs.

FIG. 11 provides the nucleotide sequence of human eIF-5A1 with exemplary siRNAs.

FIG. 12 shows a schematic of experiments showing that siRNA eIF-5A1 was able to knock down (decrease) expression in islet cells.

FIG. 13A shows the pattern of insulin secretion for 50 islets and FIG. 13B shows the pattern of [Ca2+]I from a single islet following 2.8 to 11 mM glucose stimulation. The phases are by 0, 1, 2. This figure shows a normally functioning islet responds to glucose stimulation with a biphasic pattern of insulin secretion, which closely resembles similar changes in islet intracellular calcium ([Ca²⁺]ᵢ).

FIG. 14 provides the results of calcium oscillation studies. These figures show that islet cells treated with eIF-5A1 siRNA (and thus have decreased expression of eIF-5A1 ) showed a significantly stronger glucose response and overall oscillation. This indicates that these treated cells not only survive longer but also retain functionality.

FIG. 15 shows different photographs of stained cytokine treated islet cells. The photographs shows that islet cells treated with eIF-5A1 siRNA have more live and less dead cells than the control or vehicle treated (saline only) islet cells.

FIGS. 16A and 16B show through four different imaging techniques that the siRNA gets into islets cells when administered to the islet cell donor via IP injection. Confocal images were used to generate 3D reconstructions of isolated islets. FIG. 16A shows that islets isolated from mice administered 0.9% saline intraperitoneally once daily for three days show characteristic FITC autofluorescence but minimal fluorescence in the TRITC channel (labeled Cy3 in figure). FIG. 16B shows that islets isolated from C57BI/6 mice administered Cy3-labeled stabilized siRNA intraperitoneally once daily for three days show penetrance of the Cy3 label.

FIG. 17(A) shows the results of where βTC3 cells were transfected with two different siRNA constructs (constructs A and B) or controls and subjected to immunoblotting for the indicated proteins. FIG. 17(B) shows the results of where C57B1/6 mice were injected with the same siRNAs intraperitoneally once daily for three days, and islets were isolated and purified via collagenase digestion and differential gradient centrifugation. Islets were lysed in 2% SDS and subjected to immunoblotting. FIG. 17(C) shows the results where isolated islets were loaded with Fura2 for 30 minutes before imaging in 3 mM D-glucose. Islets were stimulated with 11 mM D-glucose at ˜300 seconds, and the Fura2 ratio was continuously monitored by fluorescence microscopy. FIG. 17(D) shows the results where glucose stimulated insulin secretion (GSIS) was performed using 50 islets from each treatment group.

FIGS. 18A-I show that knockdown of eIF5A improvise islet function and survival ex-vivo.

FIGS. 19A-D show that cytokine induced of iNOS protein production is absent in eIF5A-deficient cells.

### DETAILED DESCRIPTION OF THE INVENTION

Eukaryotic initiation factor 5A (eIF5A) appears to be a crucial factor in the post-transcriptional regulation of stress-induced genes, and appears to promote cytokine-mediated apoptosis in mammalian cells. eIF5A is a small acidic protein that is very highly conserved throughout eukaryotes, and is the only protein known to contain the unique polyamine-derived amino acid hypusine. eIF5A has been proposed to play roles in mRNA processing, trafficking, and translation. However, its role, if any, in the inflammatory cascade in pancreatic islets has not before been characterized. Pancreatic islets are highly vulnerable to glucose and lipid toxicity, oxidative stress, and inflammatory cytokines. To study the role of factors involved in mediating islet to response cellular stress, the present inventors developed a protocol to deplete islets in vivo of specific proteins using small interfering RNAs (siRNAs). In a study, the inventors sought to determine the role of eIF5A in islets by depleting them of eIF5A protein using RNA interference.

However, a major challenge in RNA interference studies in primary rodent islets is their relatively poor transfection efficiency. Whereas previous work has demonstrated the utility of viral-mediated delivery of short hairpin RNAs, this technique is time-consuming to generate the appropriate viruses and is subject to viral toxicity. The inventors discovered a novel protocol to deplete mouse islets of selected proteins in vivo by repeated intraperitoneal injections of stabilized small interfering RNAs (siRNAs). This protocol results in remarkable penetration of siRNAs within the islet. This technique can be used to successfully deplete islets of any desired/target protein such as the well-characterized pancreatic transcription factor Pdx1. Using this technique, the inventors have also successfully depleted islets of eIF5A protein, and demonstrated that eIF5A contributes to cytokine-mediated islet dysfunction by promoting translation of the mRNA encoding inducible nitric oxide synthase (iNOS). The data suggest a model whereby eIF5A is necessary in pancreatic islets for stabilization and/or nuclear export of a subset of mRNAs (possibly those involved in cytokine-mediated stress responses), including iNOS mRNA.

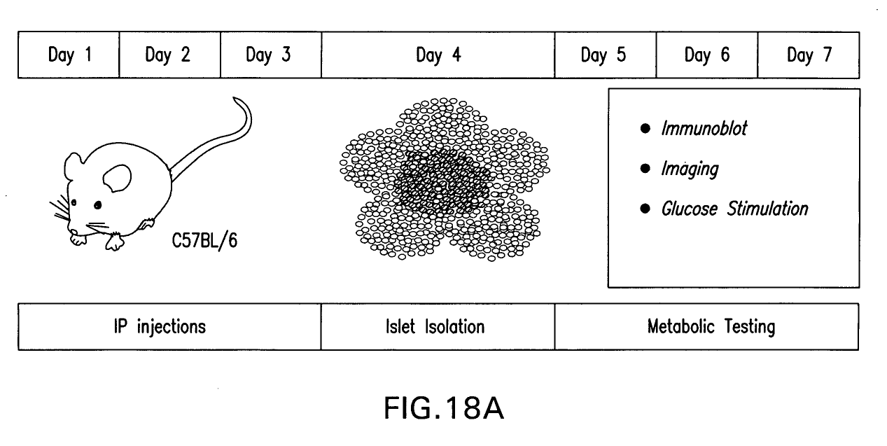

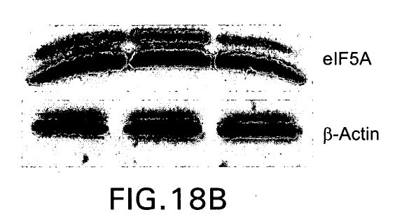

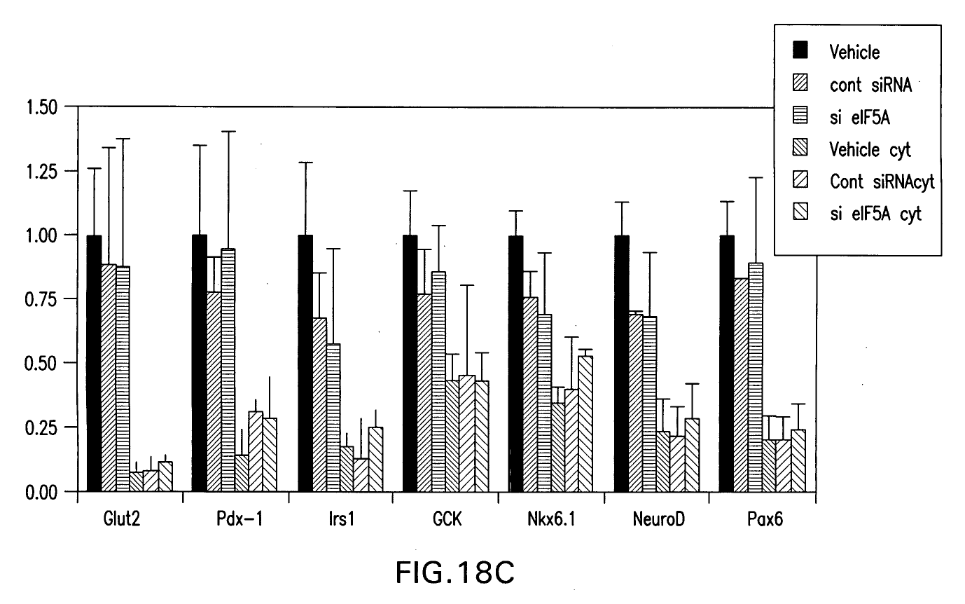

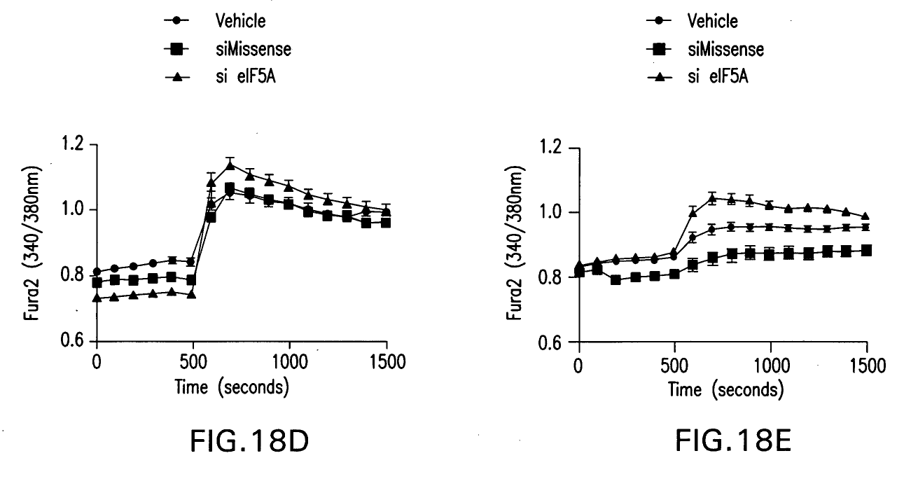

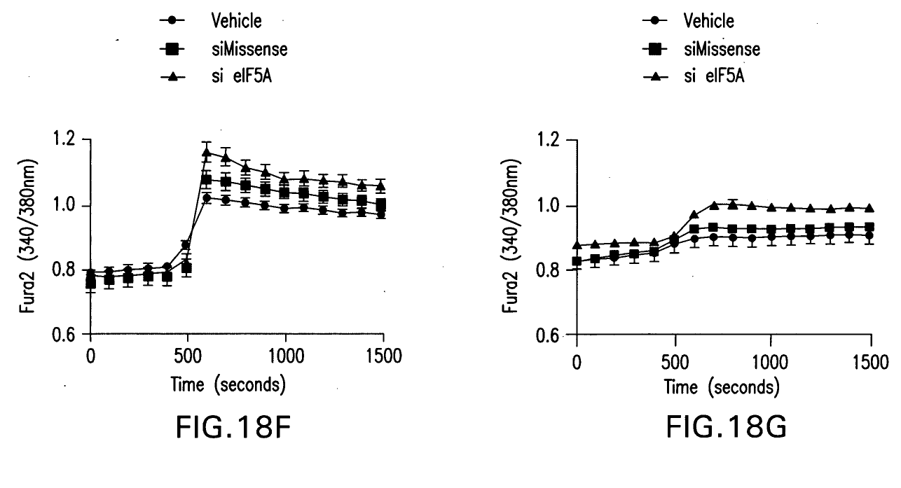

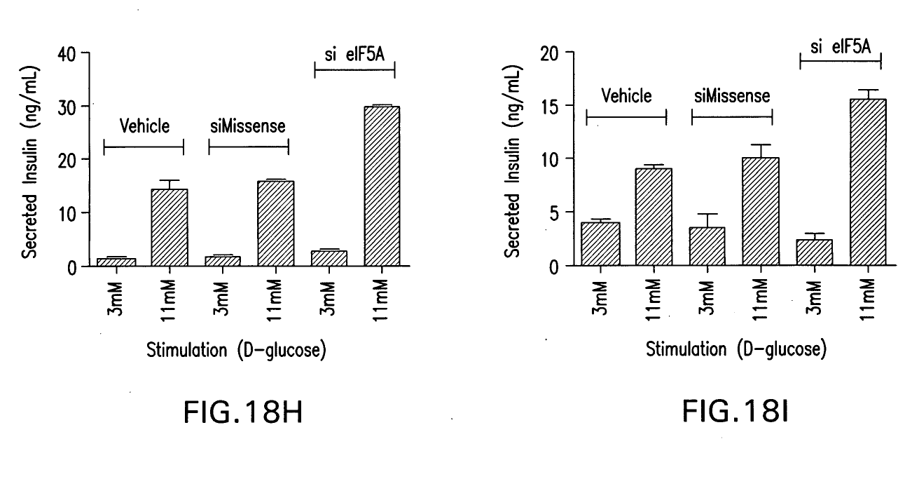

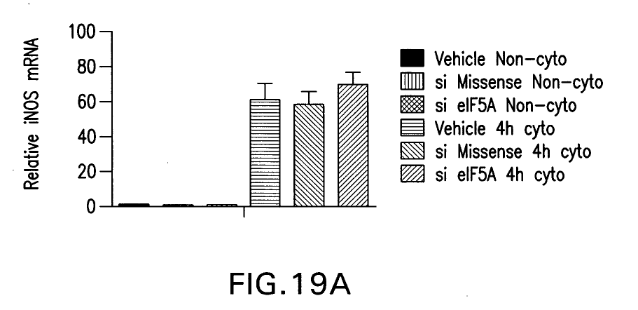

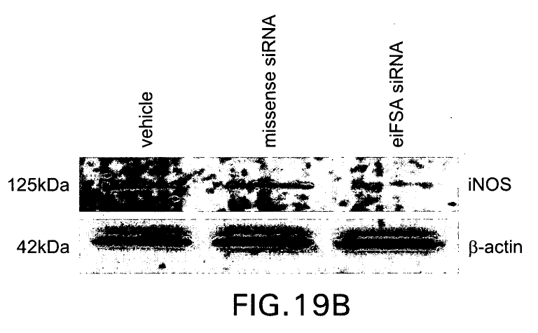

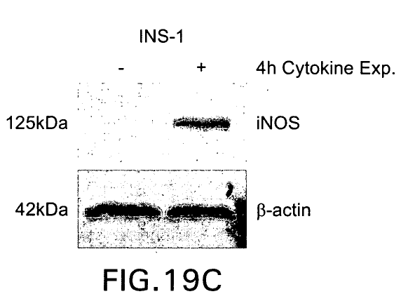

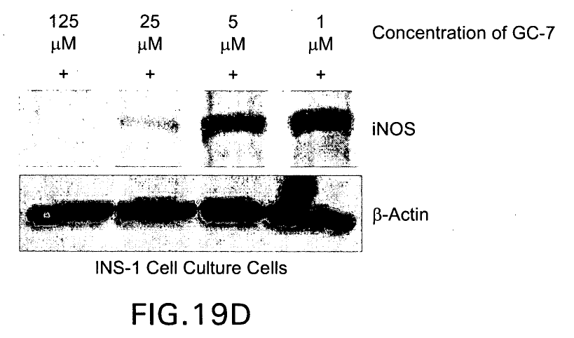

The inventors employed in vivo siRNA injection to study the role of eukaryotic initiation factor 5A (eIF5A) in cytokine-mediated stress. eIF5A has been characterized in other systems as a crucial regulator in the translation of stress-induced genes. When siRNA against eIF5A was injected into mice, a 50-70% decrease in eIF5A protein levels in isolated islets was observed. Accompanying this fall, a 30-40% increase in glucose stimulated insulin secretion and Ca2+ mobilization as compared to control treatment was observed. When isolated islets were exposed to a cocktail of proinflammatory cytokines (IL 1β, TNFα, IFNγ), the relative enhancement of islet function persisted in animals treated with si-eIF5A. Islet protection following eIF5A knockdown was not accompanied by alterations in the expression of genes whose products are involved in glucose responsiveness or insulin transcription (S1c2a2, Gck, Irs1, Nkx6-1, MafA, Pdx1, NeuroD1, and Setd7). See FIG. 18C. Importantly, although the mRNA encoding inducible nitric oxide synthase (iNOS) was upregulated more than 40-fold in both control- and si-eIF5A-treated islets in response to cytokines (see FIG. 19A), iNOS protein levels were 3-5-fold lower in si-eIF5A-treated islets (see FIG. 19B). These data suggest that enhanced function in si-eIF5A-treated islets may be secondary to reduced nitric oxide production. This provides evidence that eIF5A plays an essential role in islet β cell response to stress signals via control of iNOS translation, and as such, eIF5A may serve as a viable target for therapeutic strategies aimed at preserving viability as well as the ability of the islet cells to function after harvesting and post implantation.

The inventors have further shown that blockage of eIF-5A function via inhibition of hypusination (via administration of GC-7—an inhibitor of DHS) effectively reduces iNOS levels). See FIG. 19D.

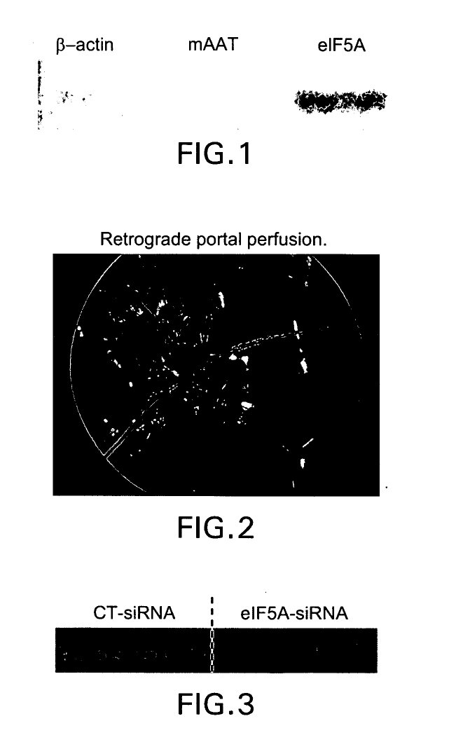

It has been previously shown that siRNA incorporation into islets can be achieved by pancreatic perfusion via retrograde portal vein inoculation. See Bradley, et al., Transplantation Proceedings, 37, 233-236, 2005. Briefly, Cy-3 labeled Luciferase (Luc) siRNA GL2 duplex was used either packaged with Lipofectamine 2000 or unpackaged, and injected either through tail vein (in vivo, 50 μg per mouse) or directly into the pancreas by retrograde portal vein inoculation (in situ, 2 μg per mouse). Pancreata were procured and stored at 4° C. for 24 hours after in situ delivery, or 4 hours after in vivo delivery, and islets were isolated and cultured an extra 16 hours before examination. To visualize siRNA distribution, pancreata were stained for insulin and examined under a fluorescent microscope. Isolated islets were directly examined under a fluorescent microscope. Unpackaged siRNA reached islets to a similar extent as observed using liposomal-packaged siRNA, agreeing with reports of so-called “naked”-siRNA delivery in vivo. Lewis et al., Nat. Genet. 32:107-108, Epub 2002 Jul. 2029, 2002 and McCaffrey A P, et al., Nature 418:38-39, 2002). FIG. 1 shows that perfusion to the islet cells provides a suitable delivery mechanism to the islet cells.

Accordingly, the present invention provides a method for inhibiting expression of a target protein in islet cells comprising administering siRNA targeted to the mRNA encoding the target protein to the islet cells, wherein the siRNA inhibits expression of the target protein in the islet cells. Administration may be through any means, preferably trough intraperitoneal administration to the islet β cell donor before the islet β cell isolation.

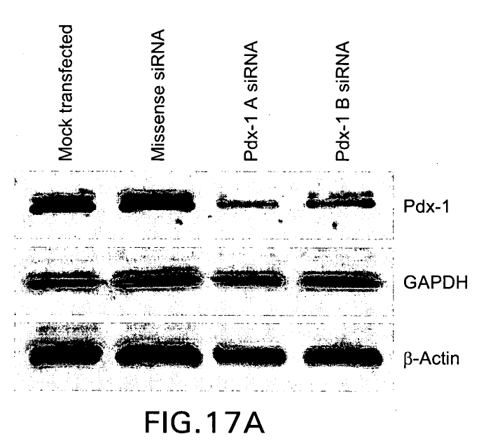

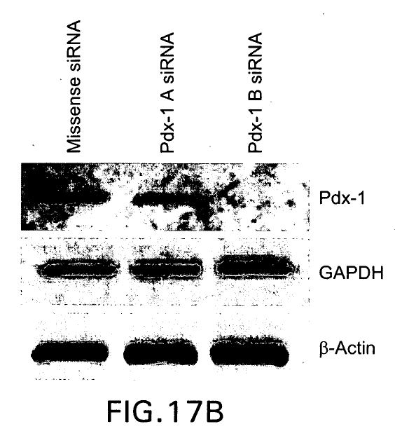

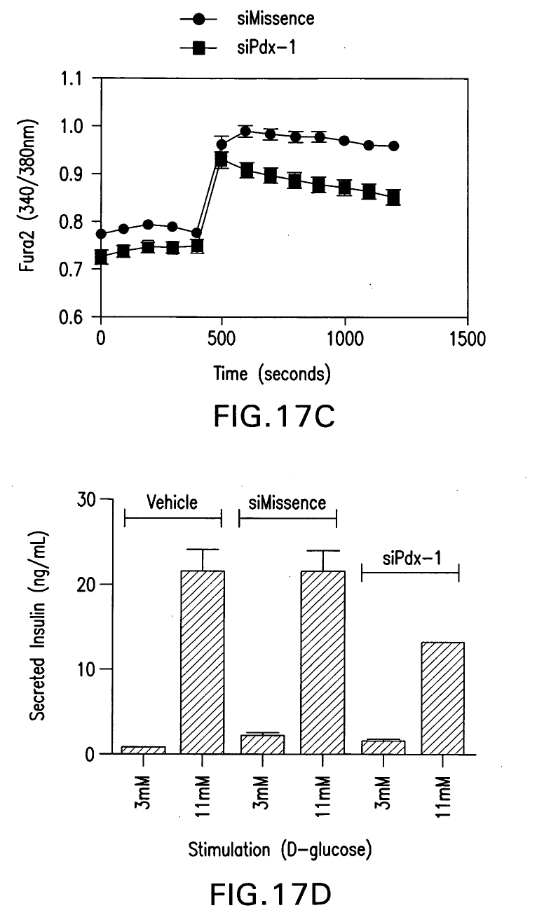

In one experiment, the inventors have shown that three daily intraperitoneal injections of stabilized, Cy3-labeled double-stranded RNAs into C57BL/6 mice revealed remarkable penetration into isolated islets, as determined by confocal microscopy. To test this technique for protein knockdown, stabilized siRNAs targeted against Pdx1 were used. Two distinct siRNAs against Pdx1 message resulted in 50% and 90% knockdown of Pdx1 protein in islets (as assessed by immunoblot analysis). See FIGS. 17A-D.

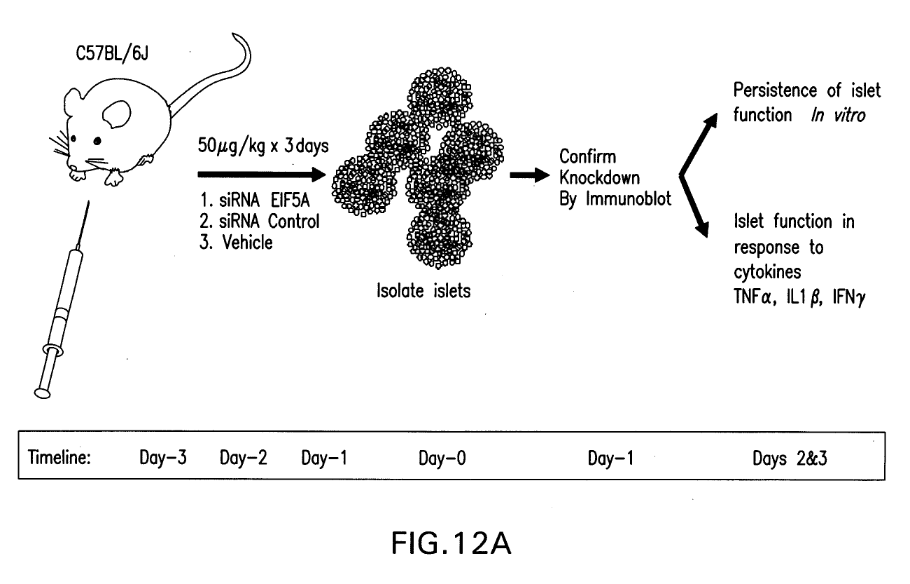

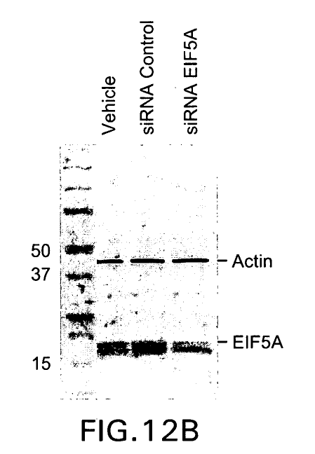

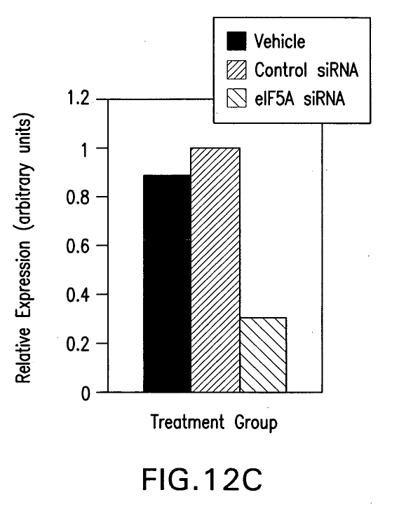

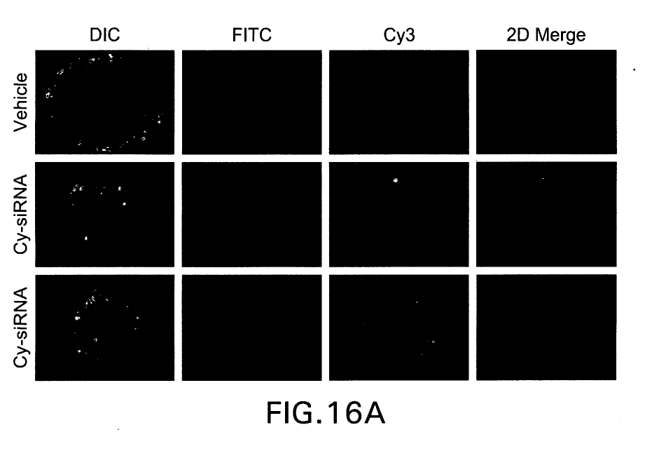

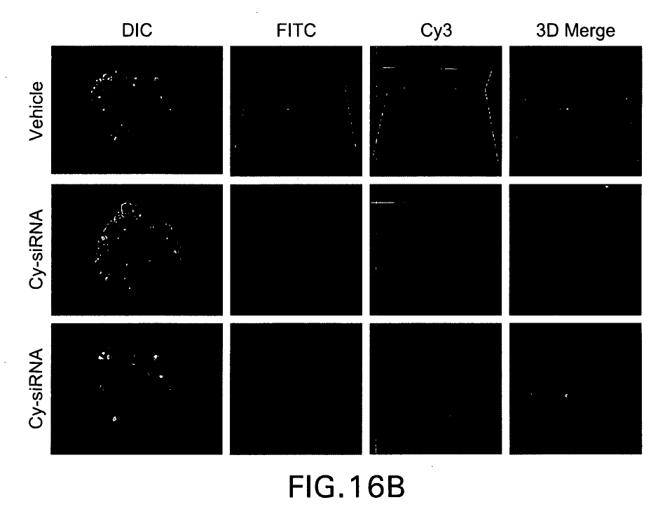

The present invention provides a method for inhibiting expression of eIF-5A1 in islet cells comprising administering eIF-5A1 siRNA to the islet cells, wherein the eIF-5A1 siRNA inhibits expression of eIF-5A1 in the islet cells. FIGS. 16A and 16B show that administration to the islet cell donor via IP injection also provides a suitable delivery mechanism to the islet cells. FIG. 16B shows that three daily intraperitoneal injections of stabilized, Cy3-labeled double-stranded RNAs into C57BL/6 mice revealed remarkable penetration into isolated islets, as determined by confocal microscopy. FIGS. 12B and 18B show that the eIF-5A1 siRNA treated islet cells do indeed express less eIF-5A1 siRNA.

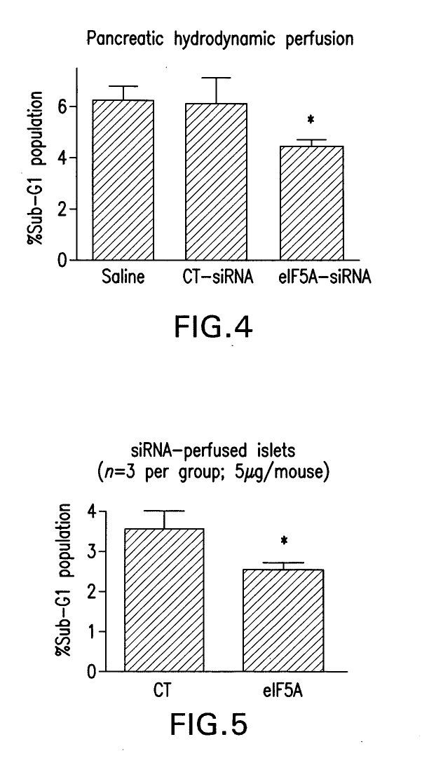

By inhibiting eIF-5A1 expression, apoptosis is also inhibited. FIGS. 4 and 5 show that treating islets cells with eIF-5A1 siRNA prior to isolation provided a mechanism to inhibit these cells from apoptosis (as demonstrated by a reduction of the number of cells in the sub-G1 phase). Accordingly, the present invention also provides a method for inhibiting apoptosis in harvested islet cells comprising administering eIF-5A1 siRNA to the islet β cell donor, wherein the eIF-5A1 siRNA inhibits expression of eIF-5A1 in the islet cells and wherein the inhibition of eIF-5A1 expression inhibits apoptosis. Preferably the islets cells are treated with eIF-5A1 siRNA cells prior to the harvesting process.

The present invention further provides a method for preserving the functionality of harvested islet cells after isolation comprising administering eIF-5A1 siRNA to the islet cells of an islet cell donor prior to islet isolation, which results in a inhibition or reduction of eIF-5A1 expression in the islet cells, which in turn inhibits apoptosis in the islet cells, as well as preserves their functionality. Preserving the functionality of the islet cells means that not only do the islet cells survive the harvesting process, but also maintain their function (the functionality of the cells remains longer and/or they maintain a better glucose response than non eIF-5A1 siRNA treated islet cells)(i.e. release insulin in response to glucose) after the harvesting process.

In one experiment, to investigate the consequence of eIF-5A1 knockdown on islet function and overall health, C57BL/6 male mice were given intraperitoneal (IP) injections of either: vehicle (0.9% saline) [Group I], control siRNA (16.6 mg/kg) [Group II], or eIF-5A1 siRNA (16.6 mg /kg) [Group III]. Injections of the assigned treatment were administered each day for three days (days −3, −2, −1) prior to islet harvest (day 0). Isolated islets were purified by collagenase treatment and allowed to rest for 16-18 hours before testing commenced. After establishing a baseline of function on Day 1 post-harvest, half of the islets were exposed to a cytokine cocktail (5 ng/mL IL1β, 10 ng/mL TNF-α, 100 ng/mL INF-γ) to mimic conditions pancreatic islets encounter in diabetes Type 1. A schematic of the experimental design and allocation of islets is depicted in FIG. 12A.

Protein lysates from each group of islets were analyzed by Western Blot for protein knockdown. FIG. 12B illustrates the relative knockdown of eIF-5A1 in Group III treated mouse islets, which is quantified in FIG. 12C. These data illustrate the efficacy of IP injection to produce knockdown expression of eIF-5A1 in islets.

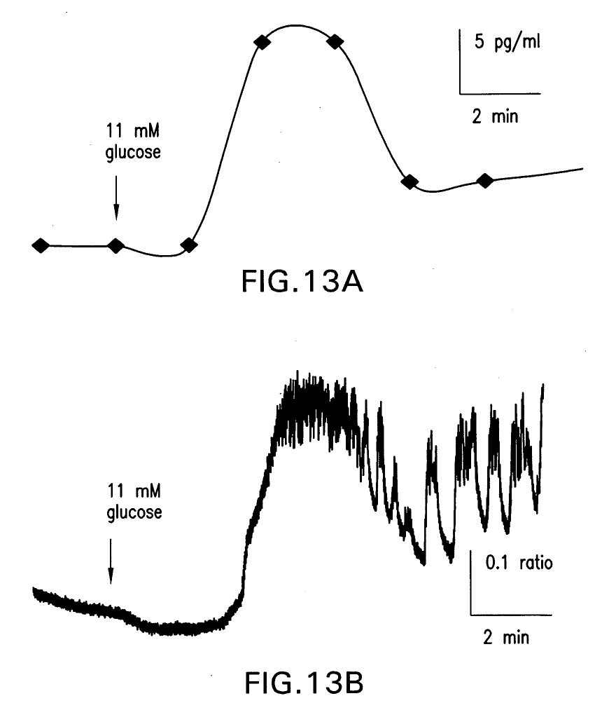

Evidence of preserved functionality of the harvested islets is demonstrated by calcium oscillation assays. The inventors have determined that intracellular calcium is an indicator of stimulation and stress. For both humans and rodents, a normally functioning islet responds to glucose stimulation with a biphasic pattern of insulin secretion, as shown in FIG. 13A. As a first approximation, this biphasic secretion pattern closely resembles similar changes in islet intracellular calcium ([Ca²⁺]ᵢ), which also consists of a first phase peak that drops into a second phase plateau (FIG. 13B). Measurements of [Ca²⁺]ᵢ reported as the ratio of emitted light from 340 and 380 nm stimulation, however, provide greater sensitivity to detect additional features of islet function. The [Ca²⁺]ᵢ response to glucose consists of three phases (see FIG. 13) that roughly reflects the processes described by the “Consensus Model” of beta-cell stimulus-secretion coupling. An initial dip in [Ca²⁺]ᵢ occurs following glucose transport into the beta-cell as the endoplasmic reticulum mobilizes and sequesters [Ca²⁺]ᵢ (phase 0). Glucose is then metabolized through glycolysis and further metabolized in the tri-carboxylic acid (TCA) cycle, which results in ATP production. When a sufficient increase in ATP/ADP occurs to close K_{ATP}-ion-channels, a sharp rise in [Ca²⁺]ᵢ occurs due to the opening of L-type calcium channels related to first phase insulin secretion. After this initial first phase peak, [Ca²⁺]ᵢ drops to a plateau that is often punctuated by [Ca²⁺]ᵢ oscillations. The plateau height is directly related to glucose concentration and the rate of second phase insulin secretion. Overall, this imaging-based [Ca²⁺]ᵢ measurement is thus a good first approximation of dynamic insulin secretion at the level of the individual islet.

Many factors can negatively impact the normal [Ca²⁺]ᵢ response to glucose stimulation. Disruptions in glucose metabolism, mitochondria and ATP production, endoplasmic reticulum (ER), ion channel function, and myriad other problems can affect the dynamics of calcium handling related to glucose stimulation. By frequently monitoring changes in [Ca²⁺]ᵢ, defects in these aspects of activity are more likely to be observed than by standard measures of static insulin secretion. For example, increased basal [Ca²⁺]ᵢ in low glucose and the loss of the phase 0 dip in [Ca²⁺]ᵢ during glucose stimulation can be indicative of ER-stress or possibly ion-channel dysfunction. Such defects can not be detected easily by measuring static or kinetic insulin secretion, but the imaging techniques described herein can be utilized to address these questions.

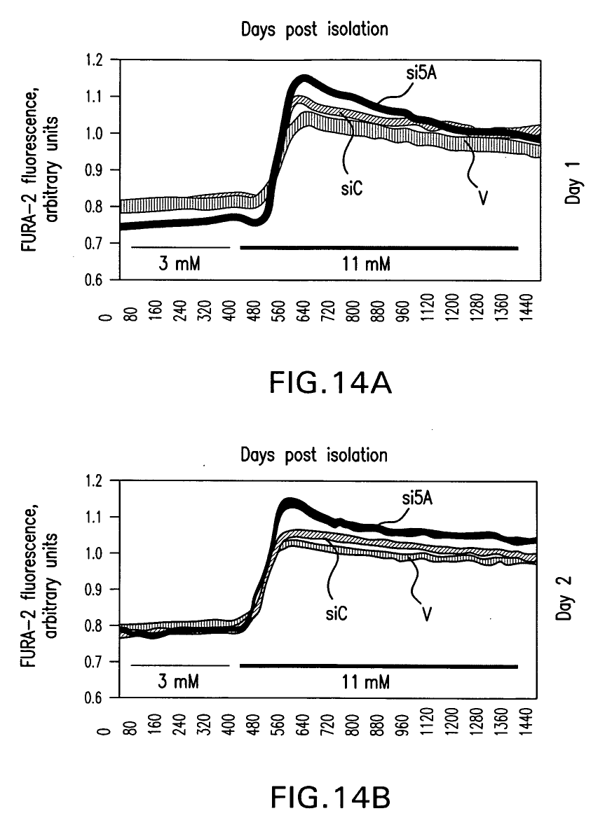

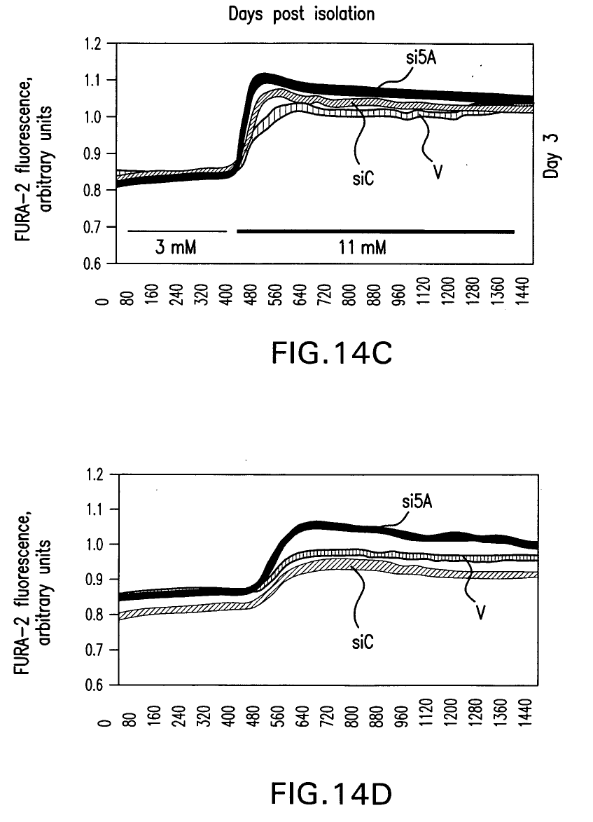

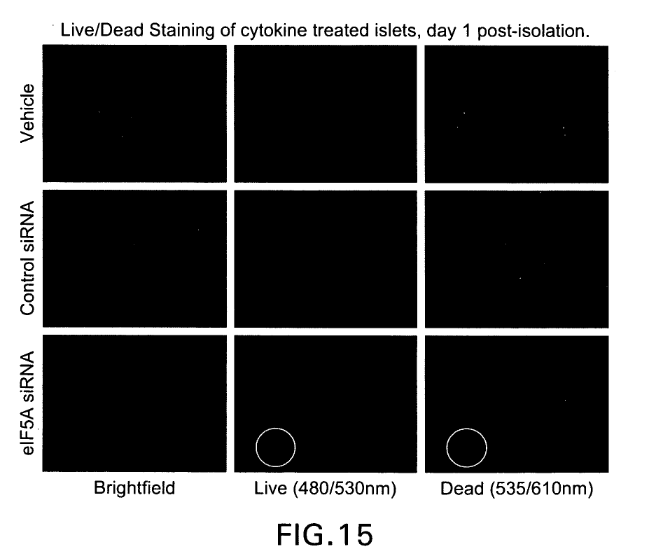

In both the presence and absence of cytokines, in calcium oscillations and glucose stimulated insulin secretions studies, the eIF-5A1 siRNA (16.6 mg/kg) treated islets [Group III (eIF-5A1 knockdown islets)] showed a greater response to glucose and an extended maintenance of functionality (implying extended survival). See FIG. 14. In accord, these islets are also expected to show a higher production of insulin (data pending). Preliminary data from images taken after Live/Dead staining qualitatively support the notion of eIF-5A1 knockdown extending the life of islet cells (FIG. 15). By comparing the number of red islets, the result of EtHD-1 intercalation into the nuclei of dead islet cells, to the number of green islets that are actively cleaving calcein-AM into the fluorescent calcein, a qualitative image of islet viability is ascertained (FIG. 15).

The present invention further provides a method of inhibiting apoptosis in cells involved in inflammatory diseases in which the disease is associated with increased production of nitric oxide. For example, inflammatory diseases such as rheumatoid arthritis (RA) are associated with increased production of nitric oxide (NO), due to activation of the inducible nitric oxide synthase (iNOS) pathway. Studies in animal models have suggested that NO plays a causal role in the pathogenesis of joint inflammation and tissue damage, since the severity of arthritis can be reduced by the administration of NOS inhibitors. Several cell types present within the joint, including synovial fibroblasts, endothelial cells and chondrocytes, can be induced by pro-inflammatory cytokines to produce NO in vitro. Moreover, localization studies have shown that there is up-regulation of iNOS expression in synovial lining cells, chondrocytes and blood vessels in joint tissues obtained from patients with RA. The localization of iNOS expression to the synovial lining layer and cartilage is of interest in the light of other studies that have shown that apoptosis is increased in RA, particularly in the synovial lining layer and cartilage. The mechanisms responsible for apoptosis in the rheumatoid joint remain unclear, although previous workers have shown that expression of Fas antigen is increased in RA synoviocytes and that Fas antibody can stimulate the apoptotic death of synoviocytes in vitro. Others have investigated the hypothesis that activation of the iNOS pathway is also involved in stimulating apoptosis in the rheumatoid joint, since high levels of NO are known to stimulate apoptosis in many cell types in vitro and have concluded that NO acts as a mediator of apoptosis in RA. See R. J. van't Hof et al., Rheumatology 200, 39:1004-1008.

As such, the present invention provides a method of inhibiting apoptosis in cells associated with RA (such as synovial and cartilage cells) by inhibiting expression of eIF5A and hence inhibits or decreases the production of nitric oxide via the iNOS pathway to inhibit apoptosis in the cells.

The present invention further provides a method of inhibiting NO production in a cell or host by administration to the cell or host an siRNA directed against eIF-5A. The siRNA decreases expression of eIF-5A and in turn decreases the production of NO by inhibiting the activation of the iNOS pathway.

The invention also provide the use of eIF5A siRNA to manufacture a medicament to decrease production of nitric oxide via the iNOS pathway.

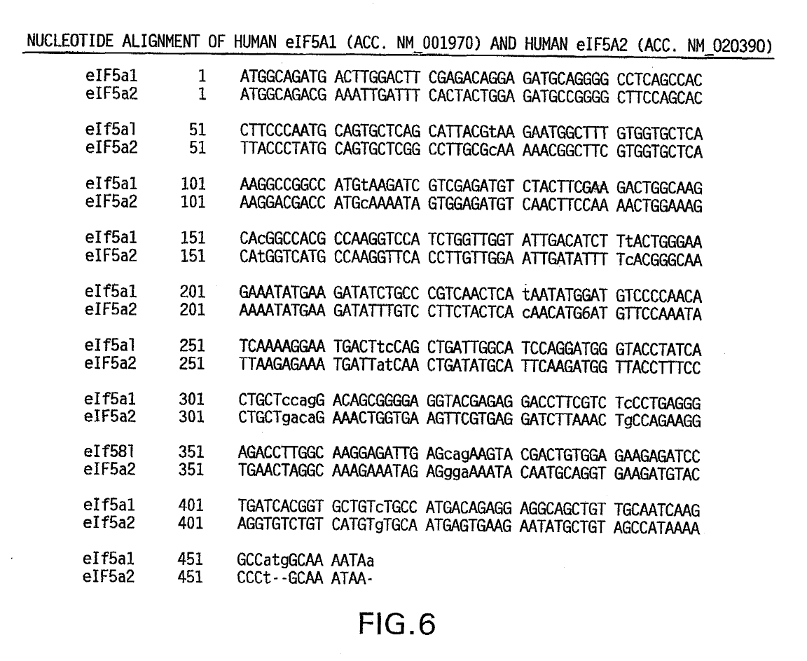

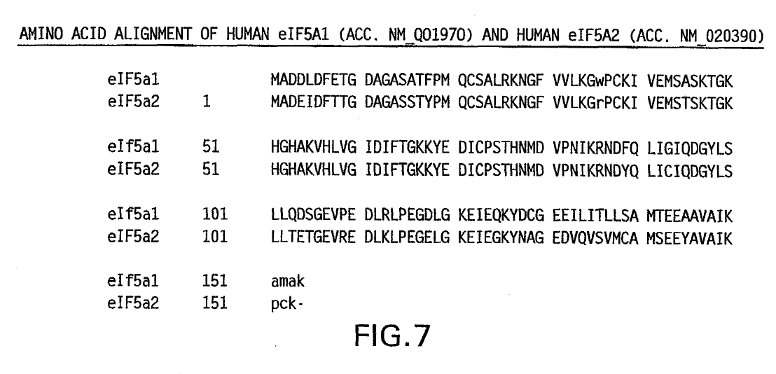

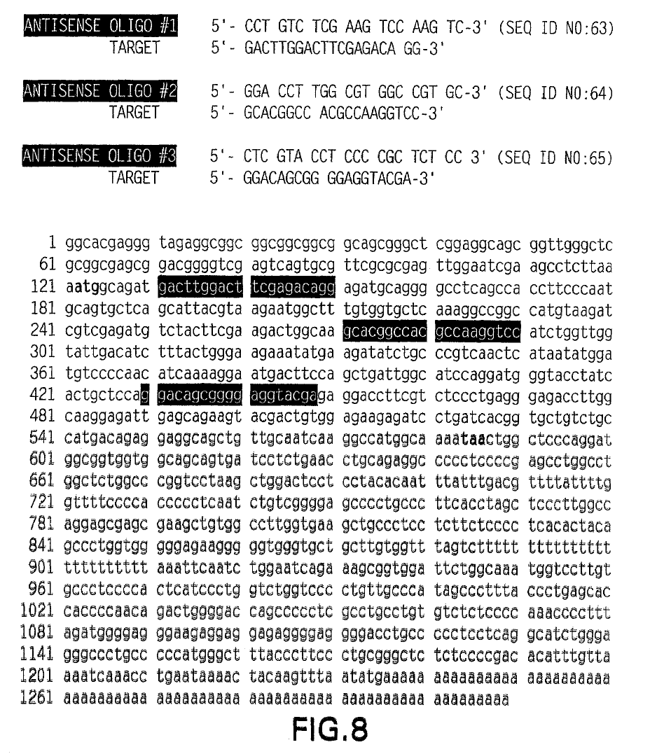

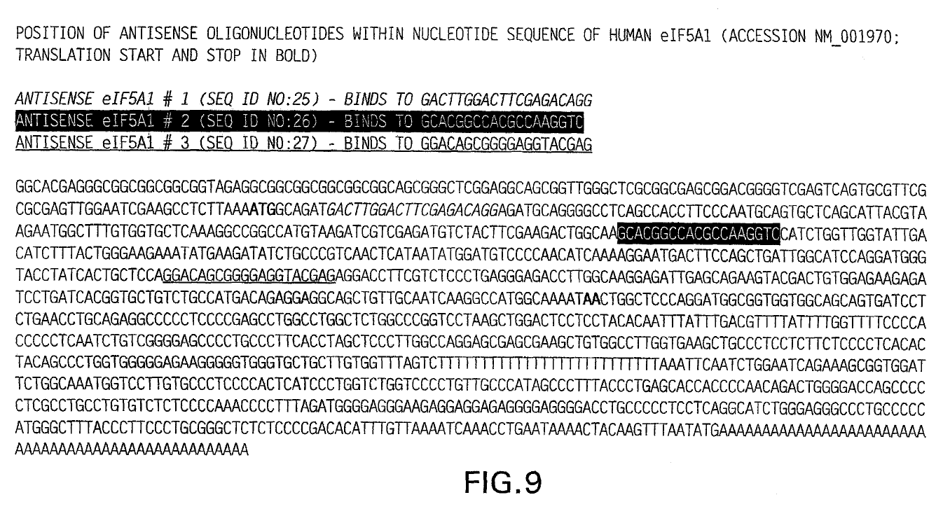

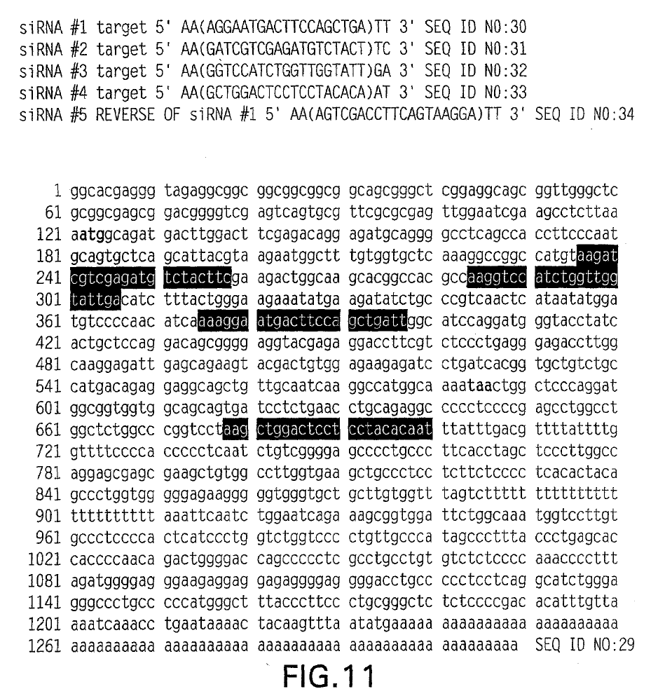

Any eIF-5A1 siRNA that inhibits expression of eIF-5A1 may be used. The term “inhibits” also means reduce as compared to levels that would occur in control cells, i.e. those cells not having been treated with eIF-5A1 siRNA. One exemplary and especially preferred eIF-5A1 siRNA comprises the sequence: 5′-AAAGGAAUGACUUCCAGCTGAdTdT-3′. Co-pending application Ser. No. 11/293,391, which was filed on Nov. 28, 2005 (which is herein incorporated by reference in its entirety) provides additional exemplary eIF-5A1 siRNAs and other antisense constructs that have been used to inhibit expression of eIF-5A1 in other cell types and were also shown to inhibit apoptosis. One skilled in the art could design other eIF-5A1 siRNAs given the eIF-5A1 sequence and can easily test for the siRNAs ability to inhibit expression without undue experimentation. FIGS. 6-11 provide sequences of eIF-5A1, exemplary eIF-5A1 siRNAs and antisense constructs. In another embodiment of the invention, antisense constructs of eIF-5A1 may be used to inhibit expression of eIF-5A1 and thus inhibit apoptosis of the islet cells, as well as maintain or preserve their functionality.

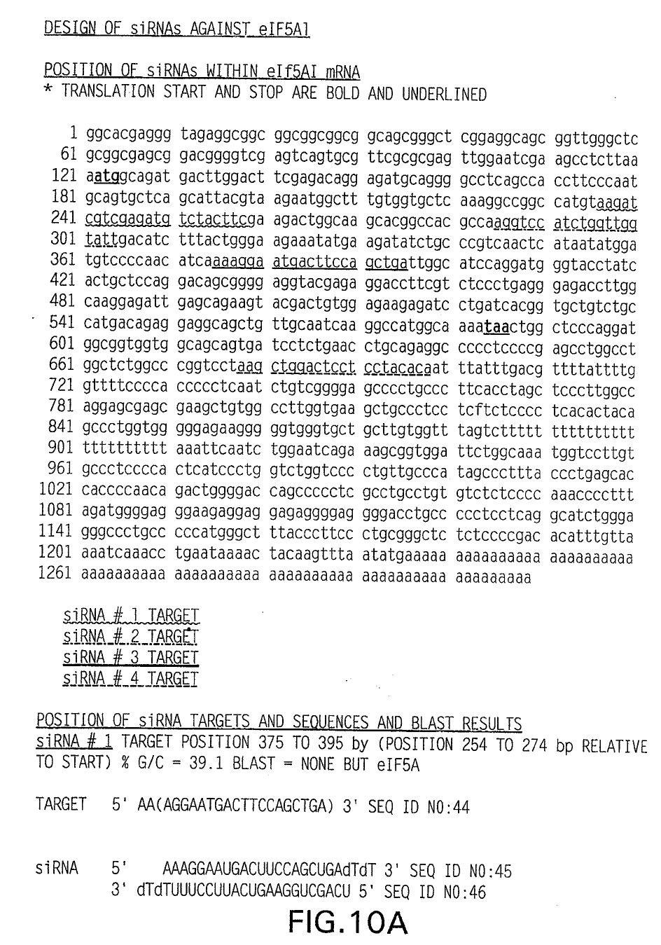

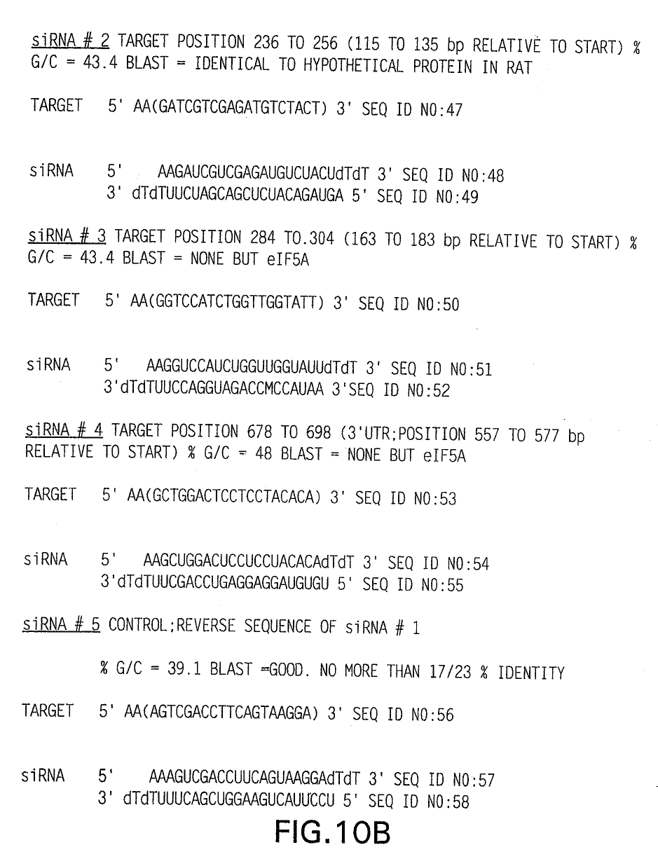

In preferred embodiments the eIF-5A1 siRNA targets the following nucleotide sequence of eIF-5A1 (see FIG. 10): 5′-AAAGGAATGACTTCCAGCTGA-3′; 5′-AAGATCGTCGAGATGTCTACT-3′; 5′-AAGGTCCATCTGGTTGGTATT-3′; and 5′-AAGCTGGACTCCTCCTACACA-3′. In especially preferred embodiments, the siRNA targets the following sequence of eIF-5A1: 5′-AAAGGAATGACTTCCAGCTGA-3′.

The present invention also provides a method for inhibiting islet cells from undergoing apoptosis during a donor harvesting process. As discussed above, many islets cells undergo apoptosis when they are harvested. The present inventors have shown that providing eIF-5A1 siRNA to the islet cells prior to harvesting, offers a protective benefit against apoptosis. The eIF-5A1 siRNA is administered to the islet cells of an islet cell donor prior to islet isolation. The donor (and hence islet cells) may be any animal, including human islet cells. Any method of administration may be used. For example, the siRNA may be administered via perfusion through the portal vein of the islet cell donor or via hydrodynamic perfusion through the portal vein of the islet cell donor. See example 1. Another form of administration includes intraperotoneal administration. See example 2 and examples 3-5.

Perfusion through portal vein is similar to canulation of the bile duct, but the needle points the opposite way. The portal vein is exposed by retraction of liver and shifting of visceral organs to the mouse's left. A preparative knot is made around it and includes the bile duct. After puncturing the vessel a blunted needle is advanced toward the pancreas and the knot is tightened around it. In a mouse model, 1 ml saline or siRNA (5 μg) is released slowly, the needle is removed and the knot is closed behind the needle to prevent fluid escape. At this point the mouse is turned around and the bile duct accessed for pancreas digestion. The pancreas may be held longer with siRNA. Alternatively, it can be removed but kept cold with collagenase longer. Regular islet isolation methods are followed and the islets (50) may be incubated in for 16 hours.

The present invention also provides a composition for inhibiting apoptosis in islet cells, comprising eIF-5A1 siRNA, wherein the siRNA inhibits expression of eIF-5A1 and thereby inhibits apoptosis and maintains the functionality of the islet cells. The composition may comprise other or additional eIF-5A1 siRNAs as discussed above. A preferred siRNA comprises the nucleotide sequence 5′-AAAGGAAUGACUUCCAGCTGAdTdT-3′

### EXAMPLES

### Example 1

### Portal Vein Perfusion

Mouse islets express eIF-5A. Total RNA was extracted from isolated mouse islets and RT-PCR was performed for (β-actin and for eIF-5A1 (FIG. 1). Resting non-stimulated islets exhibited positive levels of eIF-5A1-mRNA.

eIF-5A1-mRNA levels diminished after e1F5A1-siRNA delivery: portal vein slow perfusion. Mice were introduced 1 ml of siRNA (CT (control) sequence or eIF-5A1, 5 μg) or saline, n=2 per group, by slow retrograde portal vein perfusion (FIG. 2). Pancreata were digested by collagenase irrigation of pancreatic duct and islets were isolated as described by Lewis et al., Proc. Natl. Acad. Sci. USA, 102:12153-12158 Epub 12005 Aug. 12110, 2005. Islets (50 per mouse) were incubated for 16 hours. Total RNA was then extracted and RT-PCR was performed for β-actin and for eIF-5A1 (FIG. 3). Ratio of mRNA for eIF-5A1/β-actin was 5.24 (CT-siRNA) and 3.01 (eIF-5A1-siRNA). FIG. 3 shows that mRNA levels of eIF-5A1 were reduced in those cells treated with siRNA. This experiment was repeated with n=3 mice and islets were incubated for RNA extraction in triplicates; results were consistent with initial observation.

eIF-5A1-mRNA levels diminished and islet apoptosis rate reduced after e1F5A-siRNA delivery: portal vein hydrodynamic perfusion. Mice were introduced 1 ml of siRNA (CT or eIF-5A1, 5 μg) or saline, n=2 per group, by hydrodynamic retrograde portal vein perfusion, which was completed within 5 seconds. Pancreata were digested by collagenase irrigation of pancreatic duct and islets were isolated. Islets were incubated for 16 hours and then divided: one group was stained with propidium iodide for evaluation of apoptosis (50 islets per mouse) and the other group was processed for RT-PCR (25 islets per mouse). Levels of mRNA for eIF-5A1/β-actin were again higher in CT-siRNA group than in eIF-5A1-siRNA group. Apoptosis rate was reduced by 28.1% (FIG. 4). This experiment was repeated with n=3, apoptosis rate again diminished (FIG. 5).

Islets perfusion with biotinylated-siRNA. Biotinylated-siRNA (50 μg) was perfused into islets as described above (slow perfusion, n=1). Pancreas was fixed in formalin for staining.

siRNA. siRNA molecules were synthesized by Dharmacon, Lafayette, Colo. The sequence of the eIF-5A1 and control siRNA were: 5′ CGGAAUGACUUCCAGCUGAdTdT 3′ and 5′ AGUCGACCUUCAGUAAGGCdTdT 3′, respectively.

RT-PCR. Total RNA was extracted from cells using Qiagen RNeasy kit. eIF-5A1 Primers: Forward 5′-GAC AGT GGG GAG GTA CGA GA-3′; Reverse 5′-GGG GTG AGG AAA ACC AAA AT-3′.

Propidium iodide (PI) apoptosis stain. Single cell suspension of islets was achieved by gentle trypsinization. Cells were washed with PBS and added saponin-PI mixture containing 0.3% Saponin, EDTA 1 mM, Rnase, 1% Azide, 1% FCS and 50 μg/ml PI in PBS. Cells were thoroughly vortexed and incubated at 4° C. in the dark for 6 hours before analyzed for sub-GI population by FACS.

### Example 2

### Intraperotoneal Injection

Antibodies and siRNA: Mouse monoclonal antibody against eIF-5A1 were generated. Anti-actin monoclonal antibody (clone C4, #69100) was purchased from MP Biomedicals. In Western blots, near infrared fluorophore labeled secondary antibodies from Li-Cor were used (IRDye 800 and IRDye 700). The siRNA specific for eIF-5A1 was directed to the following sequence of eIF-5A1: 5′-AAAGGAATGACTTCCAGCTGA-3′. While control siRNA was the transcribed sequence, 5′-AAAGTCGACCTTCAGTAAGGA-3′, and was previously shown to not induce knockdown of any known protein. See co-pending U.S. applications Ser. No. 11/725,470 and PCT/US07/64424, herein incorporated by reference in their entireties.

IP (intraperotoneal) Injection of mice with siRNA: Twelve, 8- to 10-week old C57BL/6 male mice were purchased from Charles River. Male mice were chosen to avoid the metabolic variation that accompanies the female estrous cycle. Mice were randomly assigned one of three treatment groups: Group I—vehicle treatment (0.9% saline), Group II—control siRNA treatment, Group III—eIF-5A1 siRNA treatment. Each mouse received the select treatment via IP injection with 16.6 mg/kg or similar volume (in the case of Group I). Mice were injected each day (approximately 11:00 a.m.) for three days (Days −3, −2, −1) prior to islet harvest (Day 0).

Islet Isolations and Cytokine Treatment: Islet isolations from C57BL/6 mice were carried out using standard isolation techniques and a protocol approved by the Institutional Animal Care and Use Committee (IACUC). Pancreata were stored in HBSS supplemented with 4.2 mM sodium bicarbonate and 1% BSA (Invitrogen) until collagenase digestion. Purification was performed by differential centrifugation. Whole islets were maintained in phenolred-free DMEM solution supplemented with 10% FBS and 5% penicillinn/streptomycin. Cultures were incubated at 37° C. +5% CO₂ in 5.5 mM glucose. Purified islets were allowed to recover for 16-18 hours before metabolic testing (calcium oscillations).

Cytokine treatment was applied to half of the islets at the end of metabolic testing on Day 1 using a prescribed cocktail of physiologically relevant cytokines (5 ng/mL IL1β, 10 ng/mL TNT-α, 100 ng/mL INF-γ. Cytokine treated islets were allowed to rest 24 hours before further testing and protein analysis commenced (Day 2 and Day 3).

Immunoblot Assays: 10 μg of islet cell extract (prepared in Laemmli buffer containing 200 M DTT and Benzonase) were resolved by electrophoresis on a 4-20% SDS-polyacrylamide gel (Invitrogen) followed by immunoblot analysis using anti-eIF-5A1 mouse monoclonal primary antibody (at a 1:15000 dilution). Immunoblots were imaged and quantitated with the near infra red technology of the Odyssey system (Li-Cor Biosciences).

Calcium Oscillation Assays: Pancreatic islets were handpicked from collagenase-digested 8- to 10-wk-old C57BL6 mouse pancreas (as described above). Islets were treated for 20 minutes in Fura-2 low glucose (3 mM) solution then placed in a temperature-controlled flow chamber. Low glucose solution (without Fura) was flowed over the islet to establish a basal calcium measurement (5 minutes). Measurements of calcium were obtained using IP Lab 4.0 software (BD Biosciences) and are reported as the ratio of emitted light from 340 to 380 nm. After basal calcium levels were established, a high glucose (11 mM) solution was flowed through the chamber (15 minutes) to induce the cellular calcium response of the islets. The calcium response was monitored in 5-second intervals over the 20-minute period of observation. Islets are disposed of in a 5% bleach solution upon conclusion of testing.

### Example 3

### Application of siRNA Against Pdx-1 In Vitro and In Vivo

βTC3 cells were transfected with two different Pdx-1 siRNA constructs (constructs A and B) or controls and subjected to immunoblotting for the proteins Pdx-1, GAPDH and β-actin. See See FIG. 17A. C57B1/6 mice were injected with the same siRNAs intraperitoneally once daily for three days, and islets were isolated and purified via collagenase digestion and differential gradient centrifugation. Islets were lysed in 2% SDS and subjected to immunoblotting. See FIG. 17B. Isolated islets were loaded with Fura2 for 30 minutes before imaging in 3 mM D-glucose. Islets were stimulated with 11 mM D-glucose at 300 seconds, and the Fura2 ratio was continuously monitored by fluorescence microscopy. See FIG. 17C. Glucose stimulated insulin secretion (GSIS) was performed using 50 islets from each treatment group. See FIG. 17D.

### Example 4

### Knockdown of eIF-5A Improves Islet Function and Survival Ex Vivo

This example shows that in vivo administration of siRNA against eIF5A improves islet function ex vivo. FIG. 18A provides the schematic representation of experimental design. Islets were isolated post-treatment and extracts were subjected to immunoblot analysis. See FIG. 18B. Isolated islets were subjected to RT-PCR analysis of genes essential to glucose sensing and insulin transcription; although gene transcription was inhibited by cytokine treatment (4 h incubation with IFNg, IL1b, TNFa), no differences between groups were observed. See FIG. 18C. si-eIF5A-treated islets 24 h (see FIG. 18D) and 48 h (see FIG. 18F) after isolation show improved Glucose Stimulated Calcium responses (GSCa) compared to controls at 24 h (see FIG. 18D) and 48 h (see FIG. 18F) after isolatin. si-eIF5A-treated islets demonstrate enhanced GSCa in the presence of cytokines 24 h (see FIG. 18E) and 48 h (see FIG. 18G) after isolation. Islets isolated from eIF5A treated mice showed improved glucose stimulated insulin secretion (“GSIS”) compared to controls in both the absence (see FIG. 18H) and presence (see FIG. 18H) of 4 h cytokine incubation, as assessed by GSIS.

### Example 5

### Knockdown of eIF-5A Abrogates Cytokine-Induced INOS Production

Cytokine induced of iNOS protein production is absent in eIF5A-deficient cells. Islets treated with cytokines were subjected to RT-PCR for iNOS mRNA; all treatment groups showed dramatic upregulation of iNOS mRNA upon cytokine exposure. See FIG. 19A. Cytokine-treated islets were subjected to immunoblot analysis. Notably, iNOS protein was induced upon cytokine exposure in control islets, but not si-eIF5A-treated islets. See FIG. 19B. INS-1 (832/13) β cell were treated with cytokine cocktail for 4 h and subjected to immunoblotting; cells show similar increases in iNOS protein. See FIG. 19C. INS-1 cells were treated with varying concentrations of GC-7, an inhibitor of deoxyhypusine synthase, overnight, then incubated for 4 h in the presence of cytokines. INS-1 cells show an inverse correlation of iNOS production and GC-7 concentration, suggesting that inhibition of active eIF5A production prevents iNOS translation. See FIG. 19D.

### REFERENCES

1. Farrell A J, Blake D R, Palmer R M J, Moncada S. Increased concentrations of nitrite in synovial fluid and serum samples suggest increased nitric oxide synthesis in rheumatic diseases. Ann Rheum Dis 1992;51:1219-22.
2. Grabowski P S, England A J, Dykhuizen R et al. Elevated nitric oxide production in rheumatoid arthritis: detection using the fasting urinary nitrite/creatinine ratio. Eur J Pharmacol 1996;110:701-6.
3. Hilliquin P, Borderie D, Hernvann A, Menkes C J, Ekindjian O G. Nitric oxide as S-nitrosoproteins in rheumatoid arthritis. Arthritis Rheum 1997;40:1512-7.
4. McCartney-Francis N, Allen J B, Mizel D E et al. Suppression of arthritis by an inhibitor of nitric oxide synthase. J Exp Med1993;178:749-54.
5. Stefanovic-Racic M, Meyers K, Meschter C, Coffey J W, Hoffman R A, Evans C H. N-monomethyl arginine, an inhibitor of nitric oxide synthase, suppresses the development of adjuvant arthritis in rats. Arthritis Rheum1994;37:1062-9.
6. Ialenti A, Moncada S, Di Rosa M. Modulation of adjuvant arthritis by endogenous nitric oxide. Br J Pharmacol 1993;1 10:701-6.
7. Palmer R M, Hickery M S, Charles I G, Moncada S, Bayliss M T. Induction of nitric oxide synthase in human chondrocytes. Biochem Biophys Res Commun 1993;193:398-405.
8. Stadler J, Stefanovic-Racic M, Billiar T R et al. Articular chondrocytes synthesize nitric oxide in response to cytokines and lipopolysaccharide. J Immunol 1991;147:3915-20.
9. Grabowski P S, Macpherson H, Ralston S H. Nitric oxide production in cells derived from the human joint. Br J Rheum1996;35:207-12.
10. Ralston S H, Todd D, Helfrich M, Benjamin N, Grabowski P S. Human osteoblast-like cells produce nitric oxide and express inducible nitric oxide synthase. Endocrinology 1994; 135:330-6.
11. Stefanovic-Racic M, Stadler J, Georgescu H I, Evans C H. Nitric oxide synthesis and its regulation by rabbit synoviocytes. J Rheumatol 1994;21:1892-8.
12. Sakurai H, Kohsaka H, Liu M et al. Nitric oxide production and inducible nitric oxide synthase expression in inflammatory arthritis. J Clin Invest1995;96:2357-63.
13. Grabowski P S, Wright P K, van 't Hof R J, Helfrich M H, Oshima H, Ralston S H. Immunolocalisation of inducible nitric oxide synthase in the synovium and cartilage in rheumatoid arthritis and osteoarthritis. Br J Rheumatol 1997;36:651-5.
14. Firestein G S, Yeo M, Zvaifler N J. Apoptosis in rheumatoid arthritis synovium. J Clin Invest1995;96:1631-8.
15. Nakajima T, Aono H, Hasunuma T et al. Apoptosis and functional Fas antigen in rheumatoid arthritis synoviocytes. Arthritis Rheum1995;38:485-91.
16. Sioud M, Mellbye O, Forre O. Analysis of the NF-kappa B p65 subunit, Fas antigen, Fas ligand and Bc1-2-related proteins in the synovium of RA and polyarticular JRA. Clin Exp Rheumatol1998;16:125-34.
17. Brune B, Gotz C, Messmer U K, Sandau K, Hirvonen M R, Lapetina E G. Superoxide formation and macrophage resistance to nitric oxide-mediated apoptosis. J Biol Chem 1997;272:7253-8.
18. Albina J E, Cui S, Mateo R B, Reichner J S. Nitric oxide-mediated apoptosis in murine peritoneal macrophages. J Immunol 1993; 150:5080-5.
19. Blanco F J, Ochs R L, Schwarz H, Lotz M. Chondrocyte apoptosis induced by nitric oxide. Am J Pathol 1995;146:75-85.
20. Turpaev K T, Amchenkova A M, Narovlyansky A N. Two pathways of the nitric oxide-induced cytotoxic action. Biochem Mol Biol Int 1997;41:1025-33.

1. A method for preserving the functionality of harvested islet cells after isolation comprising administering eIF-5A1 siRNA to the islet cells of an islet cell donor prior to islet isolation, wherein the eIF-5A1 siRNA inhibits expression of eIF-5A1 in the islet cells and thereby inhibits apoptosis in the islet cells and preserves the functionality of the harvested islet cells.

2. The method of claim 1 wherein the eIF-5A1 siRNA targets the following nucleotide sequence of eIF-5A1: 5′-AAAGGAATGACTTCCAGCTGA-3′ (SEQ ID NO: 1); 5′-AAGATCGTCGAGATGTCTACT-3′ (SEQ ID NO: 2); 5′-AAGGTCCATCTGGTTGGTATT-3′ (SEQ ID NO: 3); or 5′-AAGCTGGACTCCTCCTACACA-3′ (SEQ ID NO: 4).

3. The method of claim 1 wherein the eIF-5A1 siRNA comprises the nucleotide sequence 5′-AAAGGAAUGACUUCCAGCTGAdTdT-3′ (SEQ ID NO: 5).

4. The method of claim 1 wherein the siRNA is administered via intraperotoneal injection to the islet cell donor.

5. A method for inhibiting islet cells from undergoing apoptosis during a donor harvesting process comprising administering eIF-5A1 siRNA to an islet cell donor via intraperotoneal injection to islet cell donor prior to islet isolation, wherein the eIF-5A1 siRNA inhibits expression of eIF-5A1 in the islet cells and thereby inhibits apoptosis in the islet cells.

6. The method of claim 5 wherein the eIF-5A1 siRNA targets the following nucleotide sequence of eIF-5A1: 5′-AAAGGAATGACTTCCAGCTGA-3′ (SEQ ID NO: 1); 5′-AAGATCGTCGAGATGTCTACT-3′ (SEQ ID NO: 2); 5′-AAGGTCCATCTGGTTGGTATT-3′ (SEQ ID NO: 3); or 5′-AAGCTGGACTCCTCCTACACA-3′ (SEQ ID NO: 4).

7. The method of claim 5 wherein the eIF-5A1 siRNA comprises the nucleotide sequence 5′-AAAGGAAUGACUUCCAGCTGAdTdT-3′ (SEQ ID NO: 5).


SEQUENCE LISTING (39 sequences)

SEQ ID NO: 1 - DNA, 21 bp, Homo sapiens
aaaggaatgacttccagctga

SEQ ID NO: 2 - DNA, 21 bp, Homo sapiens
aagatcgtcgagatgtctact

SEQ ID NO: 3 - DNA, 21 bp, Homo sapiens
aaggtccatctggttggtatt

SEQ ID NO: 4 - DNA, 21 bp, Homo sapiens
aagctggactcctcctacaca

SEQ ID NO: 5 - DNA, 23 bp, Artificial Sequence
  Description of Combined DNA/RNA Molecule Synthetic oligonucleotide
aaaggaaugacuuccagctgatt

SEQ ID NO: 6 - DNA, 21 bp, Artificial Sequence
  Description of Combined DNA/RNA Molecule Synthetic oligonucleotide
cggaaugacuuccagcugatt

SEQ ID NO: 7 - DNA, 21 bp, Artificial Sequence
  Description of Combined DNA/RNA Molecule Synthetic oligonucleotide
agucgaccuucaguaaggctt

SEQ ID NO: 8 - DNA, 20 bp, Artificial Sequence
  Description of Artificial Sequence Synthetic primer
gacagtggggaggtacgaga

SEQ ID NO: 9 - DNA, 20 bp, Artificial Sequence
  Description of Artificial Sequence Synthetic primer
ggggtgaggaaaaccaaaat

SEQ ID NO: 10 - DNA, 21 bp, Homo sapiens
aaagtcgaccttcagtaagga

SEQ ID NO: 11 - DNA, 465 bp, Homo sapiens
atggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataa

SEQ ID NO: 12 - DNA, 462 bp, Homo sapiens
atggcagacgaaattgatttcactactggagatgccggggcttccagcacttaccctatgcagtgctcggccttgcgcaaaaacggcttcgtggtgctcaaaggacgaccatgcaaaatagtggagatgtcaacttccaaaactggaaagcatggtcatgccaaggttcaccttgttggaattgatattttcacgggcaaaaaatatgaagatatttgtccttctactcacaacatggatgttccaaatattaagagaaatgattatcaactgatatgcattcaagatggttacctttccctgctgacagaaactggtgaagttcgtgaggatcttaaactgccagaaggtgaactaggcaaagaaatagagggaaaatacaatgcaggtgaagatgtacaggtgtctgtcatgtgtgcaatgagtgaagaatatgctgtagccataaaaccctgcaaataa

SEQ ID NO: 13 - protein, 154 aa, Homo sapiens [sequence omitted]

SEQ ID NO: 14 - protein, 153 aa, Homo sapiens
MADEIDFTTGDAGASSTYPMQCSALRKNGFVVLKGRPCKIVEMSTSKTGKHGHAKVHLVGIDIFTGKKYEDICPSTHNMDVPNIKRNDYQLICIQDGYLSLLTETGEVREDLKLPEGELGKEIEGKYNAGEDVQVSVMCAMSEEYAVAIKPCK

SEQ ID NO: 15 - DNA, 20 bp, Artificial Sequence
  Description of Artificial Sequence Synthetic oligonucleotide
cctgtctcgaagtccaagtc

SEQ ID NO: 16 - DNA, 20 bp, Homo sapiens
gacttggacttcgagacagg

SEQ ID NO: 17 - DNA, 20 bp, Artificial Sequence
  Description of Artificial Sequence Synthetic oligonucleotide
ggaccttggcgtggccgtgc

SEQ ID NO: 18 - DNA, 20 bp, Homo sapiens
gcacggccacgccaaggtcc

SEQ ID NO: 19 - DNA, 20 bp, Artificial Sequence
  Description of Artificial Sequence Synthetic oligonucleotide
ctcgtacctccccgctctcc

SEQ ID NO: 20 - DNA, 19 bp, Homo sapiens
ggacagcggggaggtacga

SEQ ID NO: 21 - DNA, 1309 bp, Homo sapiens
ggcacgagggtagaggcggcggcggcggcggcagcgggctcggaggcagcggttgggctcgcggcgagcggacggggtcgagtcagtgcgttcgcgcgagttggaatcgaagcctcttaaaatggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataactggctcccaggatggcggtggtggcagcagtgatcctctgaacctgcagaggccccctccccgagcctggcctggctctggcccggtcctaagctggactcctcctacacaatttatttgacgttttattttggttttccccaccccctcaatctgtcggggagcccctgcccttcacctagctcccttggccaggagcgagcgaagctgtggccttggtgaagctgccctcctcttctcccctcacactacagccctggtgggggagaagggggtgggtgctgcttgtggtttagtctttttttttttttttttttttttttaaattcaatctggaatcagaaagcggtggattctggcaaatggtccttgtgccctccccactcatccctggtctggtcccctgttgcccatagccctttaccctgagcaccaccccaacagactggggaccagccccctcgcctgcctgtgtctctccccaaacccctttagatggggagggaagaggaggagaggggaggggacctgccccctcctcaggcatctgggagggccctgcccccatgggctttacccttccctgcgggctctctccccgacacatttgttaaaatcaaacctgaataaaactacaagtttaatatgaaaaaaaaaaaaaaaaaaaaaaaaaaaaaaaaaaaaaaaaaaaaaaaaaaaaaaaaaaaaaaaaaaaaaaaaaa

SEQ ID NO: 22 - DNA, 19 bp, Homo sapiens
gcacggccacgccaaggtc

SEQ ID NO: 23 - DNA, 20 bp, Homo sapiens
ggacagcggggaggtacgag

SEQ ID NO: 24 - DNA, 1299 bp, Homo sapiens
ggcacgagggcggcggcggcggtagaggcggcggcggcggcggcagcgggctcggaggcagcggttgggctcgcggcgagcggacggggtcgagtcagtgcgttcgcgcgagttggaatcgaagcctcttaaaatggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataactggctcccaggatggcggtggtggcagcagtgatcctctgaacctgcagaggccccctccccgagcctggcctggctctggcccggtcctaagctggactcctcctacacaatttatttgacgttttattttggttttccccaccccctcaatctgtcggggagcccctgcccttcacctagctcccttggccaggagcgagcgaagctgtggccttggtgaagctgccctcctcttctcccctcacactacagccctggtgggggagaagggggtgggtgctgcttgtggtttagtcttttttttttttttttttttttttttaaattcaatctggaatcagaaagcggtggattctggcaaatggtccttgtgccctccccactcatccctggtctggtcccctgttgcccatagccctttaccctgagcaccaccccaacagactggggaccagccccctcgcctgcctgtgtctctccccaaacccctttagatggggagggaagaggaggagaggggaggggacctgccccctcctcaggcatctgggagggccctgcccccatgggctttacccttccctgcgggctctctccccgacacatttgttaaaatcaaacctgaataaaactacaagtttaatatgaaaaaaaaaaaaaaaaaaaaaaaaaaaaaaaaaaaaaaaaaaaaaaaaaaa

SEQ ID NO: 25 - DNA, 23 bp, Artificial Sequence
  Description of Combined DNA/RNA Molecule Synthetic oligonucleotide
aaaggaaugacuuccagcugatt

SEQ ID NO: 26 - DNA, 23 bp, Artificial Sequence
  Description of Combined DNA/RNA Molecule Synthetic oligonucleotide
ucagcuggaagucauuccuuutt

SEQ ID NO: 27 - DNA, 23 bp, Artificial Sequence
  Description of Combined DNA/RNA Molecule Synthetic oligonucleotide
aagaucgucgagaugucuacutt

SEQ ID NO: 28 - DNA, 23 bp, Artificial Sequence
  Description of Combined DNA/RNA Molecule Synthetic oligonucleotide
aguagacaucucgacgaucuutt

SEQ ID NO: 29 - DNA, 23 bp, Artificial Sequence
  Description of Combined DNA/RNA Molecule Synthetic oligonucleotide
aagguccaucugguugguauutt

SEQ ID NO: 30 - DNA, 22 bp, Artificial Sequence
  Description of Combined DNA/RNA Molecule Synthetic oligonucleotide
aauaccmccagauggaccuutt

SEQ ID NO: 31 - DNA, 23 bp, Artificial Sequence
  Description of Combined DNA/RNA Molecule Synthetic oligonucleotide
aagcuggacuccuccuacacatt

SEQ ID NO: 32 - DNA, 23 bp, Artificial Sequence
  Description of Combined DNA/RNA Molecule Synthetic oligonucleotide
uguguaggaggaguccagcuutt

SEQ ID NO: 33 - DNA, 23 bp, Artificial Sequence
  Description of Combined DNA/RNA Molecule Synthetic oligonucleotide
aaagucgaccuucaguaaggatt

SEQ ID NO: 34 - DNA, 23 bp, Artificial Sequence
  Description of Combined DNA/RNA Molecule Synthetic oligonucleotide
uccuuacugaaggucgacuuutt

SEQ ID NO: 35 - DNA, 23 bp, Homo sapiens
aaaggaatgacttccagctgatt

SEQ ID NO: 36 - DNA, 23 bp, Homo sapiens
aagatcgtcgagatgtctacttc

SEQ ID NO: 37 - DNA, 23 bp, Homo sapiens
aaggtccatctggttggtattga

SEQ ID NO: 38 - DNA, 23 bp, Homo sapiens
aagctggactcctcctacacaat

SEQ ID NO: 39 - DNA, 23 bp, Homo sapiens
aaagtcgaccttcagtaaggatt